(19) United States
(12) Patent Application Publication
(10) Pub. No.: US 2010/0300925 A1
(43) Pub. Date: Dec. 2, 2010

(54) MEDICAMENT CONTAINER

(76) Inventor: Lien-Ching Kan, Hsinchu Hsien (TW)

Correspondence Address: NIKOLAI & MERSEREAU, P.A., 900 SECOND AVENUE SOUTH, SUITE 820, MINNEAPOLIS, MN, 55402, US

(21) Appl. No.: 12/471,700

(22) Filed: May 26, 2009

(51) Int. Cl. B65D 83/04; A61B 19/02

(52) U.S. Cl. 206/534; 206/538

(57) ABSTRACT

A medicament container has a body and a cover. The body has multiple medicament chambers. The cover has a fastening ring and an opening device. The fastening ring is rotatably attached to the body and has multiple labels. The opening device is capable of opening or closing the medicament chambers and has a through hole for accessing the medicament chambers. When the medicament in one medicament chamber is consumed, the opening device is rotated to access the next medicament chamber and indicates respective label. Thus a user can approximately estimate the amount of remaining medicaments in the medicament container at a glance.

### BACKGROUND OF THE INVENTION

1. Field of the Invention

The present invention relates to a medicament container, especially to a medicament container for keeping solid medicaments such as tablets or capsules.

2. Description of the Prior Art

A conventional medicament container comprises a body and a cover. The body comprises an inner space for storing medicaments. The cover covers the body to seal the inner space of the body and may be opened to access the medicaments.

Multiple medicaments such as pills, tablets or capsules are filled in the inner space of the body. A user, usually according to a prescription, takes a determined amount of medicaments in a determined time period that may be a day or a few hours. However, the conventional medicament container does not indicate the amount of remaining medicaments. The user may not aware that medicaments are totally spent until he or she opens the cover to take medicaments for the next time period. The user may be forced to seek new medicament supply for the time period. The user may also have to skip the medicament he or she supposed to take in the price of risking his or her health.

To overcome the shortcomings, the present invention provides a medicament container to mitigate or obviate the aforementioned problems.

### SUMMARY OF THE INVENTION

The main objective of the invention is to provide a medicament container.

The medicament container in accordance with the present invention has a body and a cover. The body has multiple medicament chambers. The cover has a fastening ring and an opening device. The fastening ring is rotatably attached to the body and has multiple labels. The opening device is capable of opening or closing the medicament chambers and has a through hole for accessing the medicament chambers.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a medicament container in accordance with the present invention;

FIG. 2 is another perspective view of the medicament container in FIG. 1;

FIG. 3 is an exploded perspective view in partial section of a second embodiment of the medicament container in accordance with the present invention;

FIG. 4 is a side view in partial section of the medicament container in FIG.

3;

FIG. 5 is a side view in partial section of a third embodiment of the medicament container in accordance with the present invention; and

FIG. 6 is a top view of a fifth embodiment of the medicament container in accordance with the present invention.

### DETAILED DESCRIPTION OF THE PREFERRED EMBODIMENTS

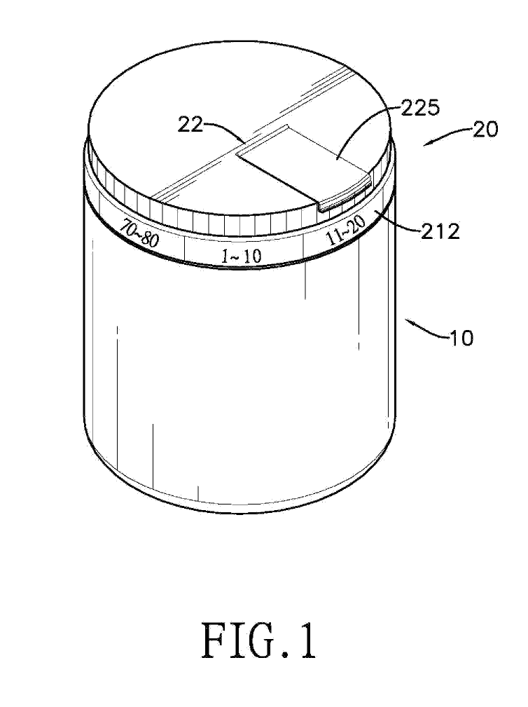

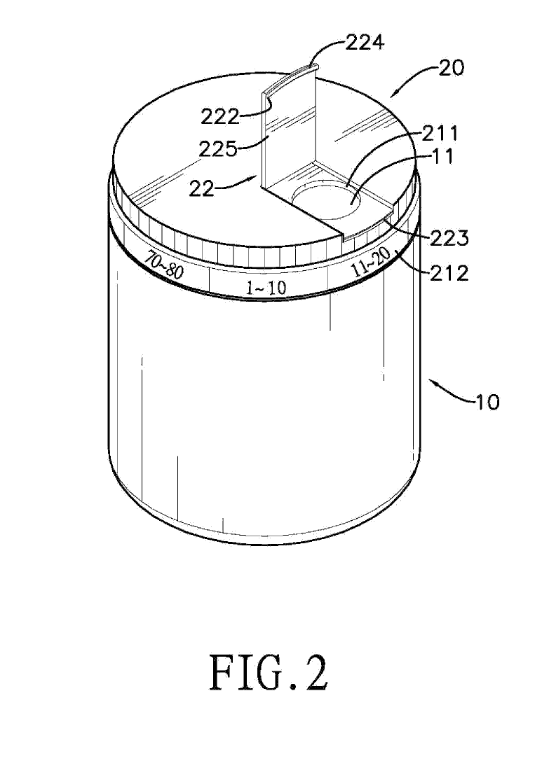

With reference to FIGS. 1 and 2, a medicament container in accordance with the present invention comprises a body (10) and a cover (20). The body (10) comprises a body top, a body bottom and multiple medicament chambers (11). Each medicament chamber (11) is inwardly recessed on the body top and into the body (10) and comprises a chamber opening. The chamber opening is formed on the body top of the body (10) so that the medicament chamber (11) may be filled with pills, tablets or capsules through the chamber opening.

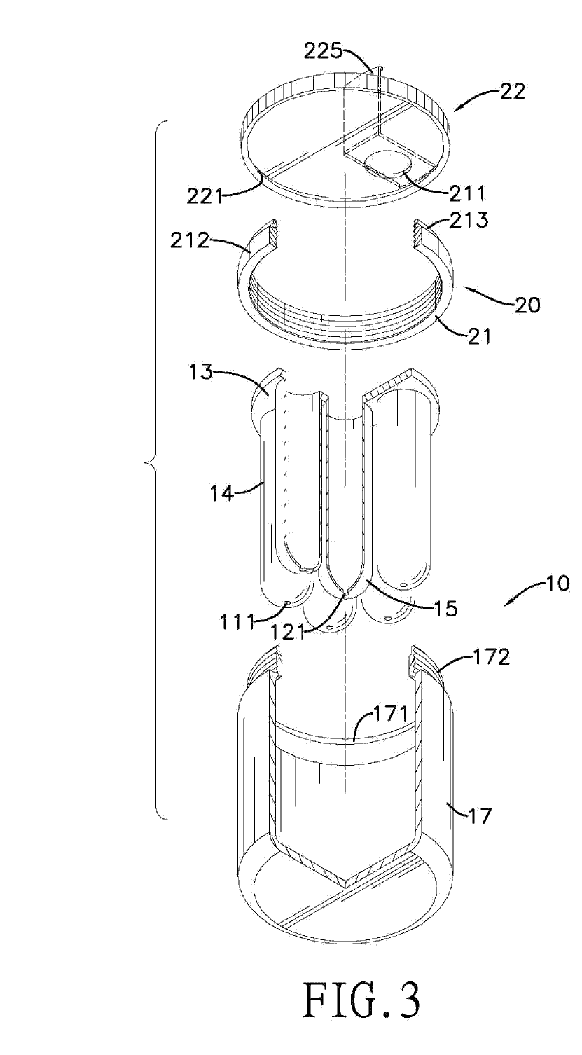

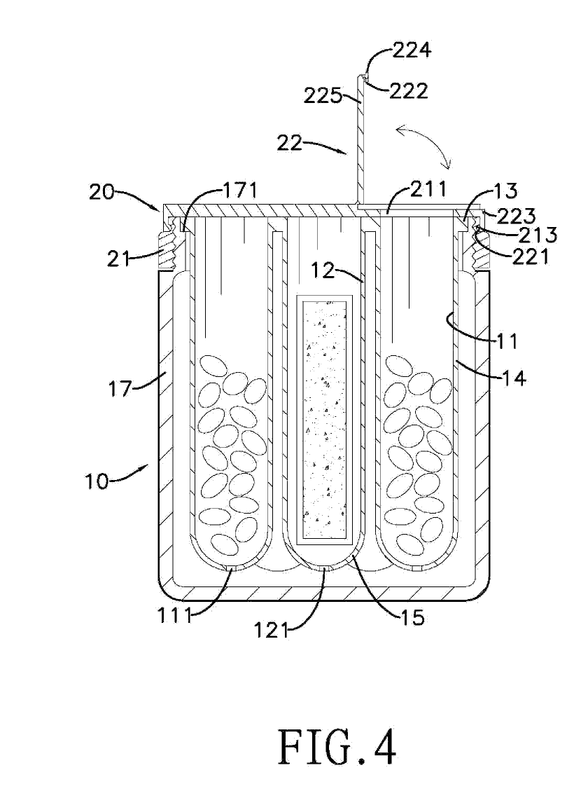

With further reference to FIGS. 3 and 4, the cover (20) comprises a fastening ring (21) and an opening device (22).

The fastening ring (21) is rotatably attached to the body top of the body (10, 10A) and comprises multiple labels (212). The multiple labels (212) correspond to the multiple medicament chambers (11, 11A).

The opening device (22) is capable of opening or closing the chamber openings of the multiple medicament chambers (11, 11A) and comprises a through hole (211). The through hole (211) corresponds to the chamber openings of the multiple medicament chambers (11, 11A). Medicaments such as pills, tablets or capsules may be stored and effectively managed according to the labels (212).

With further reference to FIGS. 3 and 4, in a first embodiment of the present invention, the body (10) comprises a housing (17), a disk (13) and multiple tubes (14). The housing (17) comprises a housing top, a containing space and a housing opening formed on the housing top. The disk (13) covers the housing opening and sealing the housing (17). The multiple tubes (14) are fastened to the disk (13) and extend into the containing space of the housing (17). In addition, the aforementioned multiple medicament chambers (11) are respectively formed inside the tubes (14) and the chamber openings of the multiple medicament chambers (11) are respectively formed on the disk (13).

In the first embodiment, it is preferred that the fastening ring (21) of the cover (20) is rotatably attached to the body top of the body (10) and fastens the disk (13) in the housing opening of the housing (17). In such structure, the disk (13) fastened in the housing opening of the housing (17) holds the multiple tubes (14) within the containing space of the housing (17).

In a practical structure for fastening the disk (13) and attaching the fastening ring (21), the fastening ring (21) comprises multiple corresponding rings formed on an inner surface of the fastening ring (21). In addition, the housing (17) further comprises a groove (171) and multiple engaging rings (172). The groove (171) is formed on an inner surface of the housing (17) near the housing opening. The groove (171) is used to engage an outer edge of the disk (13) to fasten the disk (13) to the housing opening. The engaging rings (172) are formed on an outer surface of the housing (17) near the housing opening and engage with the corresponding rings of the fastening ring (21). The engagement of the engaging rings (172) with the corresponding rings allows the fastening ring (21) to be rotatably attached to the body top of the body (10). The fastening ring (21) is mounted on the housing opening of the housing (17), fasten the disk (13) and thus help hold the tubes (14) within the containing space of the housing (17).

The opening device (22) is attached to a top of the fastening ring (21). The opening device (22) is used to open or close the through hole (211) by fastening to or releasing from the fastening ring (21). The fastening ring (21) may further comprise an annular groove (213) formed on an outer surface of the fastening ring (21). The opening device (22) is an annular disk attached to the top of the fastening ring (21). The opening device (22) comprises an outer rim, an annular protrusion (221), an engaging groove (223) and a lid (225). The annular protrusion (221) is formed on the outer rim of the opening device (22) and protrudes towards the fastening ring (21) to engage with the annular groove (213) and fasten the opening device (22) to the fastening ring (21).

The engaging groove (223) is formed on an outer surface of the opening device (22) and radially corresponds to the through hole (211). The lid (225) comprises a distal end, a proximally bent hook (222) and an optional boss (224). The hook (222) is formed at the distal end and is used to engage with the engaging groove (223). The boss (224) is formed at the distal end of the lid (225) and protrudes outwards. A user can easily control the lid (225) to open or close the through hole (211) by pushing the boss (224) to engage the hook (222) with or release the hook (222) from the engaging groove (223).

When using the present invention, the medicament chambers (11) are filled with an appropriate amount of medicaments such as pills, tablets or capsules. The opening device (22) is rotated to move the through hole (211) over a chamber opening of one of the medicament chambers (11) to access the medicaments received in that chamber (11). When the medicaments in that chamber (11) are spent, the fastening ring (21) is rotated to move the through hole (211) over the chamber opening of the next medicament chamber (11). By rotating the opening device (22), the through hole (211) is moved over the medicament chambers (11) to access medicaments. The position of the lid (225) indicates respective labels (212) of the medicament chambers (11) so that the user can approximately estimate the amount of remaining medicaments in the medicament container in accordance with the present invention. Since the amount of remaining medicaments is obvious at a glance, the user can easily decide when and how much supplying medicaments to purchase.

It is known that some medicaments have to be stored in a dry environment. A desiccant agent is often used to create a suitable dry environment for medicament storage. To conveniently use desiccant agents in the medicament container in accordance of the present invention, each medicament chamber (11) may further comprise a ventilation hole (111) and the body (10) may further comprise a desiccant chamber (12). The desiccant chamber (12) is inwardly recessed from the body top into the body (10) and comprises a desiccant opening and an aeration hole (121). The desiccant opening is formed on the body top of the body (10). The aeration hole (121) ventilates with the ventilation holes (111) of the multiple medicament chambers (11). Desiccant agents are put in the desiccant chamber (12). With the ventilating aeration hole (121) and ventilation holes (111), the desiccant agents are able to create a dry environment within the medicament chambers (11) and the desiccant chamber (12). As a result, the medicaments in the medicament chambers (11) would be stored in a suitable dry environment.

In a second embodiment the body (10) further comprises a desiccant tube (15) that is fastened to the disk (13) and extends into the containing space of the housing (17). The desiccant chamber (12) is formed inside the desiccant tube (15). The desiccant opening of the desiccant chamber (12) is formed on the disk (13).

In addition, the ventilation holes (111) of the medicament chambers (11) are respectively formed through a tube wall of the medicament tubes (14). The aeration hole (121) of the desiccant chamber (12) is formed through a tube wall of the desiccant tube (15). Within the housing (17), desiccant agents that are put in the desiccant chamber (12) create a dry environment that is suitable for medicament storage.

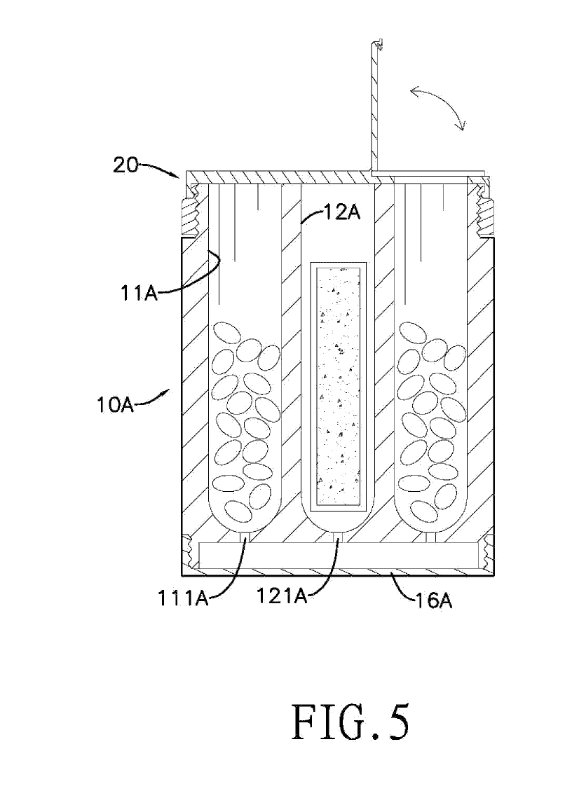

With reference to FIG. 5, in a third embodiment in accordance with the present invention, the body (10A) is a hollow cylinder and comprises a body top, a body bottom and multiple medicament chambers (11A). Each medicament chamber (11A) is inwardly recessed on the body top and into the body (10A) and comprises a chamber opening. The chamber opening is formed on the body top of the body (10A).

In addition, the ventilation holes (111A) of the medicament chambers (11A) penetrate through the body bottom of the body (10A). The aeration holes (121A) of the desiccant chambers (12A) penetrate through the body bottom of the body (10A). The body (10A) further comprises a base (16A). The base (16A) is attached beneath the body bottom the body (10A) and comprises a ventilation space (161A). The ventilation space (161A) allows the aeration hole (121A) to ventilate with the ventilation holes (111A) of the multiple medicament chambers (11A).

The ventilation space (161A) communicates with the aeration hole (121A) and the ventilation holes (111A) and thus desiccant agents put in the desiccant chamber (12A) provides suitable storage environment for the medicaments stored in the medicament chambers (11A).

A fourth embodiment, without the desiccant chamber (12A), the base (16A) and the ventilation holes (11A), is similar to the third embodiment. Medicaments need not to be stored in a dry environment can be stored in the fourth embodiment of the medicament container in accordance invention.

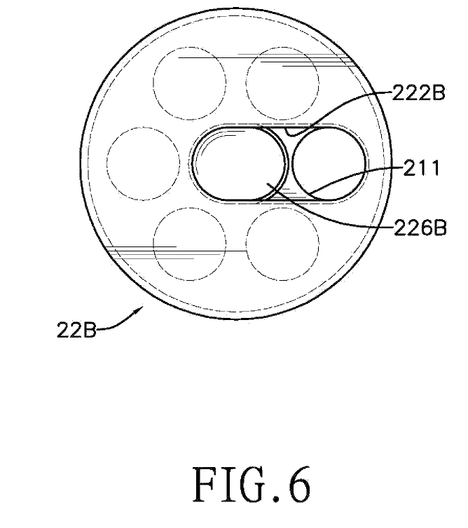

With reference to FIG. 6 and further reference to FIGS. 3 and 4, in a fifth embodiment of the medicament container of the present invention, the opening device (22B) being attached to the top of the fastening ring (21) as described above and comprises an elongated hole (222B) and a sliding lid (226B). The elongated hole (222B) is located over the through hole (211). The sliding lid (226B) is slidably mounted in the elongated hole (222B). The through hole (211) may be opened or closed by sliding the sliding lid (226B).

With the aforementioned medicament container in accordance with the present invention, a user can approximately estimate the amount of remaining medicaments in the medicament container at a glance.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

Following is a list of the symbols used above.

10 body

10A body

11 medicament chamber

11A medicament chamber

111 ventilation hole

111A ventilation hole

11A ventilation hole

12 desiccant chamber

121 aeration hole

121A aeration hole

12A desiccant chamber

13 disk

14 medicament tube

15 desiccant tube

161A ventilation space

16A base

17 housing

171 groove

172 engaging rings

20 cover

21 fastening ring

211 through hole

212 label

213 annular groove

22 opening device

221 annular protrusion

222 hook

222B elongated hole

223 engaging groove

224 boss

225 lid

226B sliding lid

22B opening device

What is claimed is:

1. A medicament container comprising
a body comprising
a body top;
a body bottom; and
multiple medicament chambers and comprising
a chamber opening formed on the body top of the body; and a cover comprising
a fastening ring and comprising multiple labels; and
an opening device and comprising a through hole.

2. The medicament container as claimed in claim 1, wherein each medicament chamber; and
the body further comprises
a desiccant chamber and comprising
a desiccant opening formed on the body top of the body; and an aeration hole.

3. The medicament container as claimed in claim 1, wherein
the body comprises
a housing comprising
a housing top;
a containing space; and
a housing opening formed on the housing top;
a disk; and
multiple tubes;
the multiple medicament chambers; and
the chamber openings of the multiple medicament chambers.

4. The medicament container as claimed in claim 2, wherein
the body comprises
a housing comprising
a housing top;
a containing space; and
a housing opening formed on the housing top of the housing;
a disk; and
multiple medicament tubes;
the multiple medicament chambers;
the chamber openings of the multiple medicament chambers;
the body further comprises
a desiccant tube;
the desiccant chamber;
the desiccant opening of the desiccant chamber;
the ventilation holes; and
the aeration hole.

5. The medicament container as claimed in claim 2, wherein
the ventilation holes;
the aeration holes; and
the body further comprises
a base and comprising
a ventilation space allowing the aeration hole.
